(19) United States
(12) Patent Application Publication
(10) Pub. No.: US 2025/0025028 A1
(43) Pub. Date: Jan. 23, 2025

(54) Multichannel Integrated Endoscope System

(71) Applicant: OmniVision Technologies, Inc., Santa Clara, CA (US)

(72) Inventors: Jau-Jan Deng, Taipei City (TW); Chang-Long Chen, Taoyuan City (TW); Shih-Hsin Hsu, Taipei City (TW)

(21) Appl. No.: 18/354,318

(22) Filed: Jul. 18, 2023

(51) Int. Cl. A61B 1/05; A61B 1/06

(52) U.S. Cl. CPC A61B 1/051; A61B 1/0676

(57) ABSTRACT

A multichannel endoscope system includes a light module configured to emit illumination light on an object, an image module configured to capture an image of the object, and a multichannel sensor module configured to obtain a spectral information of the object. The multichannel sensor module includes an image lens, a light homogenizer and a multichannel array sensor, where the light homogenizer is formed between the multichannel array sensor and the image lens.

### TECHNICAL FIELD

The present invention relates to technology field of endoscope system, and more particularly, a multichannel integrated endoscope system.

### BACKGROUND

An endoscope is a medical tool for being inserted into a human body to provide observation of organs or body cavity in which a lesion may not be directly observed without an operation or incision.

As image processing technology continuously evolved, initially a black and white camera was replaced by a color image device to form a color endoscope system used to capture images of each part in the body cavity, and thus a lesion in each part may be examined in detail through captured images. As depicted in FIG. 1(A), a conventional color endoscope imaging system 100 contains a lighting module 101 and an imaging module 103 having an image lens and an image sensor, where the lighting field of view (FOV) 101a and imaging FOV 103a are respectively illustrated. The lighting module provides light source(s) to illuminate biological tissues. When a biological tissue is irradiated with light, light reflected by the surface of the biological tissue can be collected by the image lens and then be imaged by the image sensor. FIG. 1(B) illustrates image sensor layout of the conventional color endoscope imaging system 100. The color image sensor utilizes three color filters, i.e. R, G, B, having respectively different spectral transmittance characteristics, as shown in FIG. 1(C), to pass three light bands. Signal of each channel and white balance setting are computed based on the responses of the color filters. The color endoscope imaging system shown in FIG. 1(A) exhibits good spatial resolution in imaging but inadequate spectral information.

In order to improve the poor performance of getting spectral information of the irradiated object by the endoscope system shown in FIG. 1, a single-zone spectral sensor is integrated into the endoscope system. The endoscope system 200 with a single-zone spectral sensor is illustrated in FIG. 2(A), which includes a lighting module 201, an imaging module 203 and a single-zone spectral sensor module 205, where the lighting field of view (FOV) 201a and imaging FOV 203a are respectively illustrated. The single-zone spectral sensor module 205 shown in FIG. 2(B) includes a mechanical opening 205-1, a homogenizer (diffuser) 205-2 and a multichannel sensor 205-3 having a plurality of channels. FIG. 2(C) and FIG. 2(D) respectively show the filter layout of the multichannel sensor and spectra of channels, for example, Blue (B), Green (G), Yellow (Y), Red (R) and gray (g) channels. Typically, a diffuser is used as the homogenizer to increase the uniformity of irradiance on the spectral sensor. The full width at half maximum (FWHM) of each channel's spectrum, shown in FIG. 2(D), is generally narrower than that of the conventional color image sensors. White Balance settings for the endoscope system 200 are computed based on the responses of the spectral sensor. Design of endoscope system shown in FIG. 2 can obtain good spatial resolution in imaging but only get average spectral information of the captured scene.

To further improve the performance of getting spectral information of the irradiated object by the endoscope system shown in FIG. 2, a multichannel array sensor is proposed and integrated into the endoscope system. The endoscope system 300 with a multichannel array sensor is illustrated in FIG. 3(A), the multichannel array sensor is used to replace the single-zone spectral sensor, which includes a lighting module 301, an imaging module 303 and a multichannel sensor module 305, where the lighting field of view (FOV) 301a, imaging FOV 303a and sensing FOV 305a are respectively illustrated. The multichannel sensor module 305 shown in FIG. 3(B) includes an imaging lens 305-1, a light homogenizer 305-2 and a multichannel array sensor 305-3. The multichannel array sensor 305-3 as illustrated in FIG. 3(C) has a plurality of zones 30 and each zone has a plurality of channels, for example, Blue (B), Green (G), Yellow (Y), Red (R) and gray (g) channels, there are gaps between adjacent zones for reducing crosstalk from adjacent sub-FOVs, where FOV is stand for field of view. The full width at half maximum (FWHM) of each channel's spectrum as shown in FIG. 3(D) is generally narrower than that of conventional color image sensors. A diffuser is used as the light homogenizer to increase the uniformity of irradiance within individual zone. However, this newer design of endoscope system utilized a diffuser bulk, the irradiance on multichannel array sensor 305-3 shown in FIG. 3(E) can still result poor uniformity of the outer zones due to the non-uniform relative illuminance and low transmittance caused by the appearance of a diffuser.

In view of various technical issues of the aforementioned conventional endoscope systems, there is a need to provide an endoscope system capable of improving these deficiencies.

### SUMMARY OF THE INVENTION

In one aspect, the present invention provides a multichannel integrated endoscope system, which includes a light module configured to emit illumination light on an object, an image module configured to capture an image of the object, and a multichannel sensor module configured to obtain a spectral information of the object. The multichannel sensor module includes an image lens, a light homogenizer and a multichannel array sensor, where the light homogenizer is formed between the multichannel array sensor and the image lens.

In another aspect, the present invention provides a multichannel sensor module, which includes an imaging lens configured to receive light reflected from an object, a light homogenizer configured to homogenize the light reflected from the object, and a multichannel array sensor configured to receive light reflected from the object for obtaining spectral information of the object. The light homogenizer is formed between the multichannel array sensor and the imaging lens.

### BRIEF DESCRIPTION OF THE DRAWINGS

The components, characteristics and advantages of the present invention may be understood by the detailed descriptions of the preferred embodiments outlined in the specification and the drawings attached:

FIG. 1(A) shows an exemplary endoscope system according to a prior art.

FIG. 1(B) and FIG. 1(C) respectively illustrate image sensor color filter layout and spectra of the color filter utilized in the endoscope system shown in FIG. 1(A).

FIG. 2(A) shows other exemplary endoscope system according to a prior art.

FIG. 2(B) illustrates the single-zone spectral sensor module of the endoscope system shown in FIG. 2(A).

FIG. 2(C) and FIG. 2(D) respectively illustrate image sensor color filter layout and spectra of the color filter utilized in the single-zone spectral sensor module shown in FIG. 2(B).

FIG. 3(A) illustrates the other exemplary endoscope system according to a prior art.

FIG. 3(B) illustrates the multichannel sensor module of the endoscope system shown in FIG. 3(A).

FIG. 3(C) and FIG. 3(D) respectively illustrate multichannel array sensor layout and spectra of the color filter utilized in individual zone of the multichannel array sensor of the multichannel sensor module shown in FIG. 3(B).

FIG. 3(E) illustrates irradiance on multichannel array sensor of the multichannel sensor module shown in FIG. 3(B) at various zone locations.

FIG. 4(A) illustrates a multichannel integrated endoscope system according to an embodiment of the present invention.

FIG. 4(B) depicts a schematical drawing showing that sub-FOVs of an object (or region of interest) can be mapped onto corresponding spectral sensing zones on sensor plane through the imaging lens according to an embodiment of the present invention.

FIG. 4(C) illustrates a multichannel sensor module of the endoscope system shown in FIG. 4(A) according to an embodiment of the present invention.

FIG. 4(D) and FIG. 4(E) respectively illustrate multichannel array sensor layout and spectra of the color filter utilized in individual zone of the multichannel array sensor of the multichannel sensor module shown in FIG. 4(C).

FIG. 5(A) illustrates a cross-sectional view of a light homogenizer with single side microlens array according to an embodiment of the present invention.

FIG. 5(B) illustrates a cross-sectional view of a light homogenizer with single side microlens array according to another embodiment of the present invention.

FIG. 5(C) illustrates a front view of a light homogenizer with microlens array according to an embodiment of the present invention.

FIG. 5(D) schematically illustrates light path of the imaging lens being projected onto the multichannel array sensor according to an embodiment of the present invention.

FIG. 6 illustrates irradiance on multichannel array sensor of the multichannel sensor module shown in FIG. 4(C) at various zone locations according to an embodiment of the present invention.

FIG. 7(A) and FIG. 7(B) are respectively illustrating axial light path and off-axial light path of microlens array of the light homogenizer according to embodiments of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

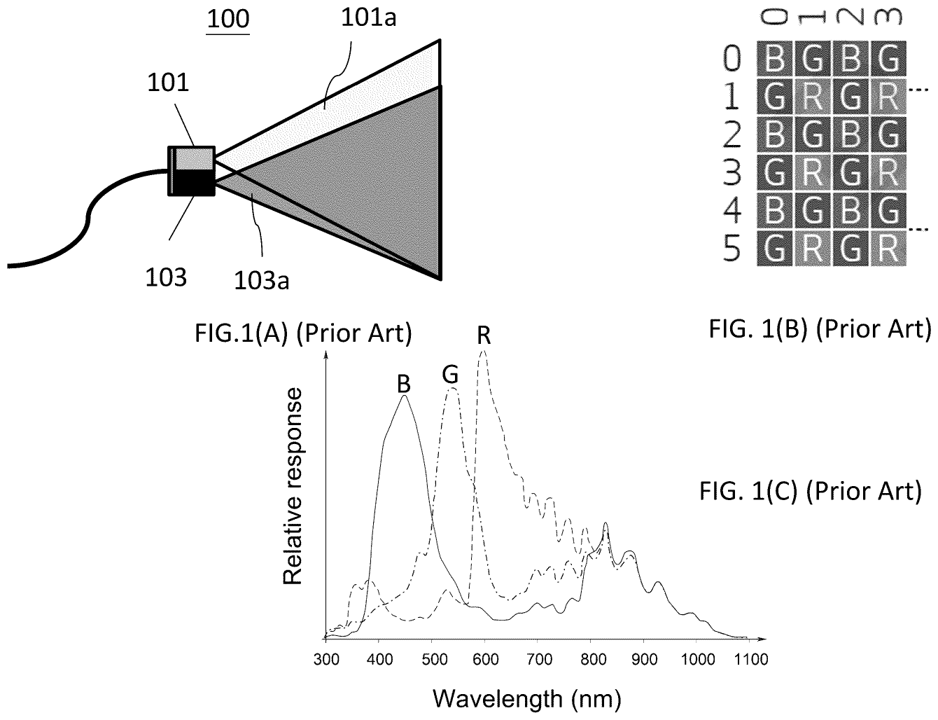

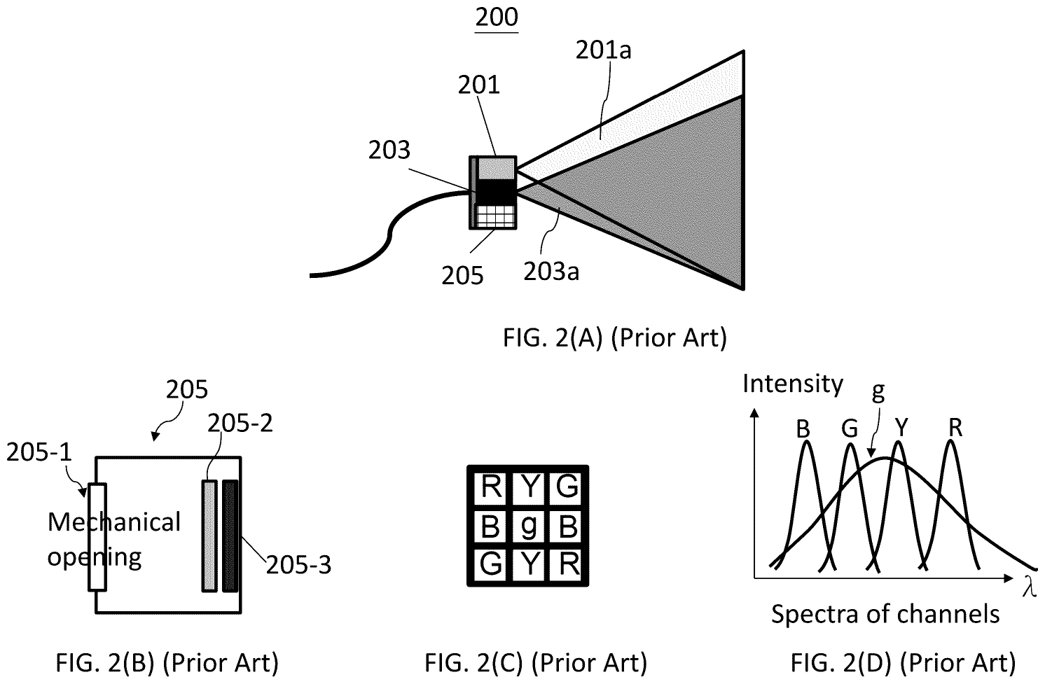

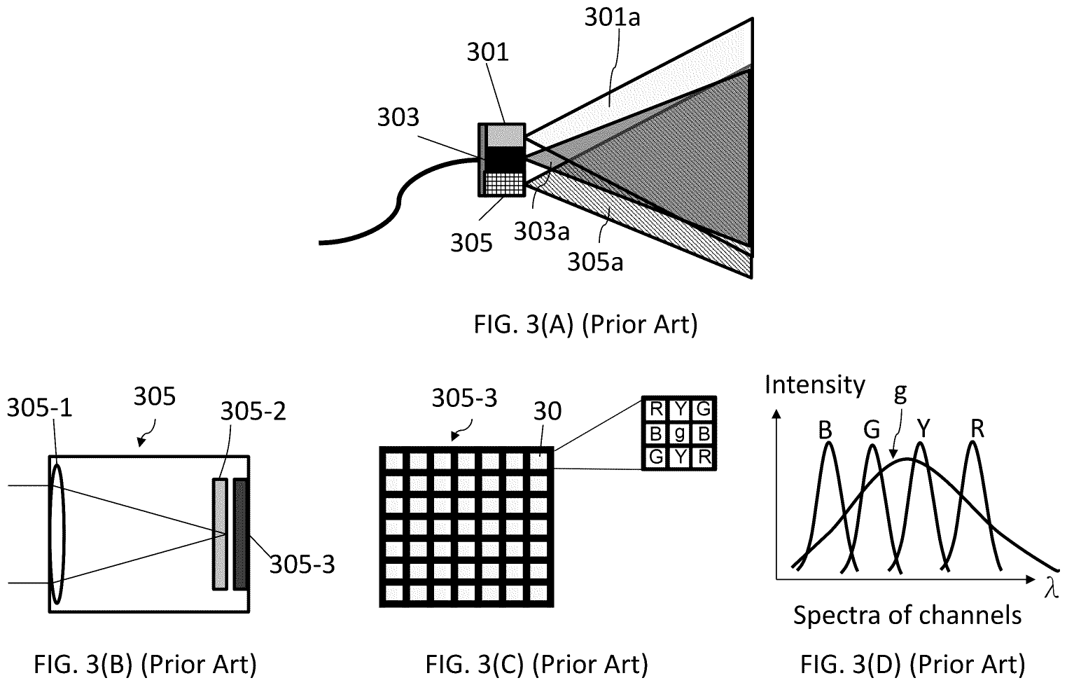

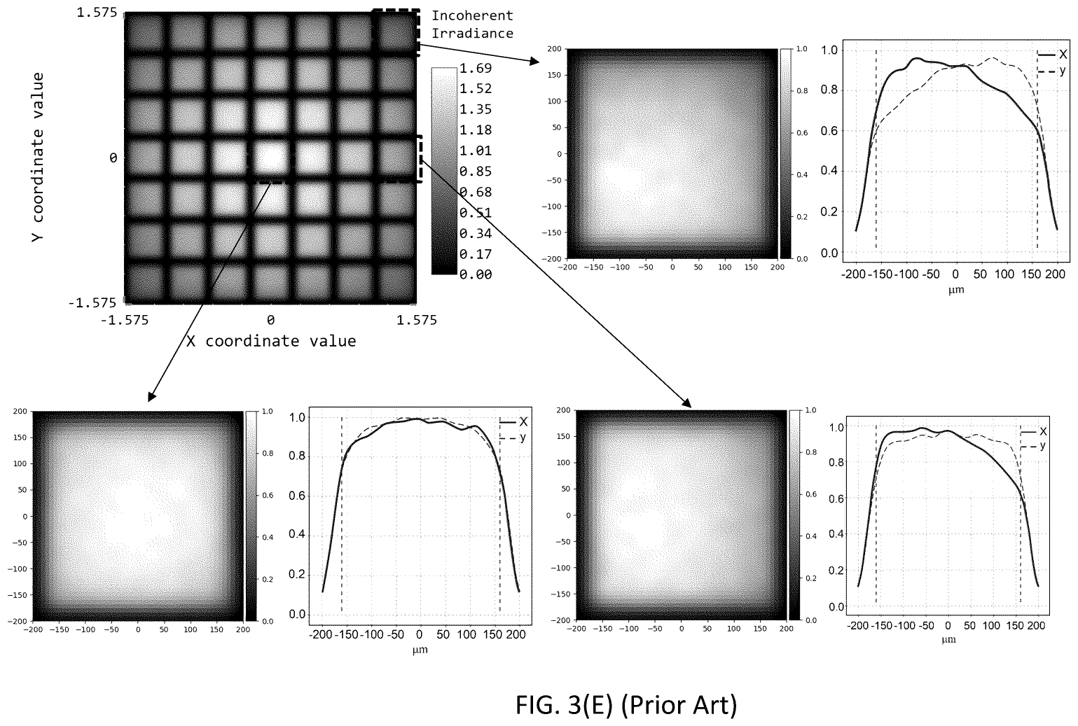

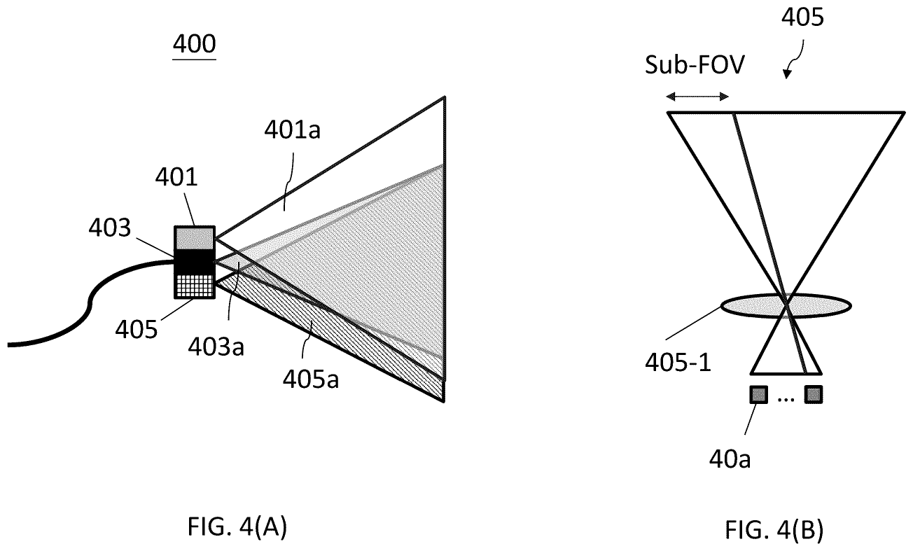

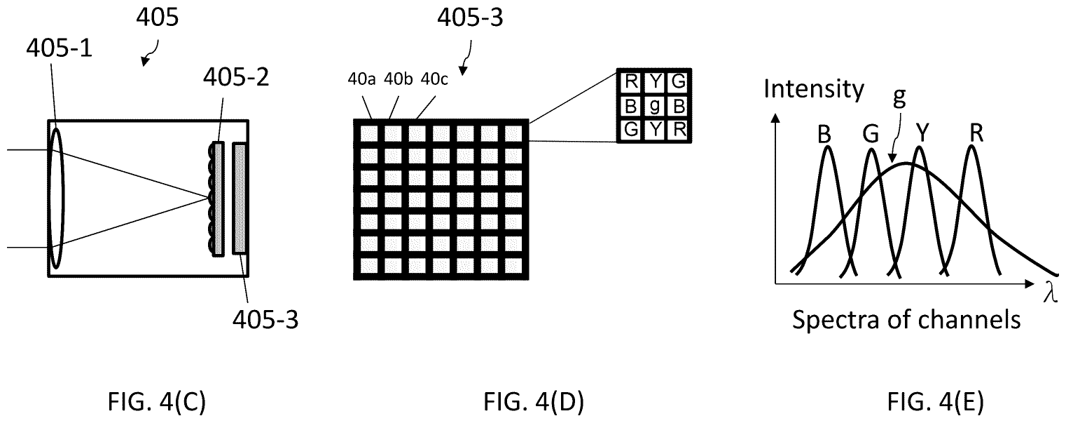

To solve the issues, such as inadequate spectral information, inadequate color information under mixed lighting condition, low transmittance and poor uniformity due to the use of conventional diffusor, encountered by the conventional endoscope systems mentioned in FIGS. 1-3, the present invention proposes a multichannel integrated endoscope system 400, as shown in FIG. 4(A), including a lighting module 401, an imaging module 403 and a multichannel sensor module 405, where the lighting field of view (FOV) 401a, imaging FOV 403a and sensing FOV 405a are respectively illustrated. The lighting module 401 is configured to emit illumination lights on an object. The imaging module 403 includes an imaging lens and an image sensor configured to capture images of the object. The multichannel sensor module 405 configured to obtain spectral information of the object through an image lens, a light homogenizer and a multichannel array sensor, details will be described in the subsequent paragraphs. FIG. 4(B) depicts a schematical drawing showing that sub-FOVs of an object (or region of interest) can be mapped onto corresponding spectral sensing zones (40a, . . . ) on sensor plane through the imaging lens, where individual zone is one of a plurality spectral sensing zones of a multichannel array sensor of the multichannel sensor module 405.

According to embodiments of the present invention, as depicted in FIG. 4(C), the multichannel sensor module 405 adapts an image lens 405-1, a microlens array (MLA) 405-2 to act a light homogenizer and a multichannel array sensor 405-3 together configured to sense field of view (FOV) of an object.

According to embodiments of the present invention, as shown in FIG. 4(D), the multichannel array sensor 405-3 includes a plurality of spectral sensing zones (40a, 40b, 40c, . . . ) arranged to form an spectral sensing array and each spectral sensing zone has a plurality of spectral-filter channels, for examples Blue (B), Green (G), Yellow (Y), Red (R) and Gray (g), each zone divides the sensing FOV into sub FOVs and each sub-FOV is corresponded to a specific spectral sensing zone.

According to embodiments of the present invention, there are gaps between adjacent zones for reducing crosstalk from adjacent sub-FOVs, where FOV is stand for field of view. The full width at half maximum (FWHM) of each channel's spectrum shown in FIG. 4(E) is generally narrower than that of conventional color image sensors.

According to embodiments of the present invention, FOV of the imaging lens of the multichannel sensor module at least covers the FOV of the imaging lens of the imaging module.

According to embodiments of the present invention, FOV of the imaging lens of the multichannel sensor module is between 90 and 120 degrees.

According to embodiments of the present invention, F-number of the imaging lens of the multichannel sensor module is between 1.4 and 5.6.

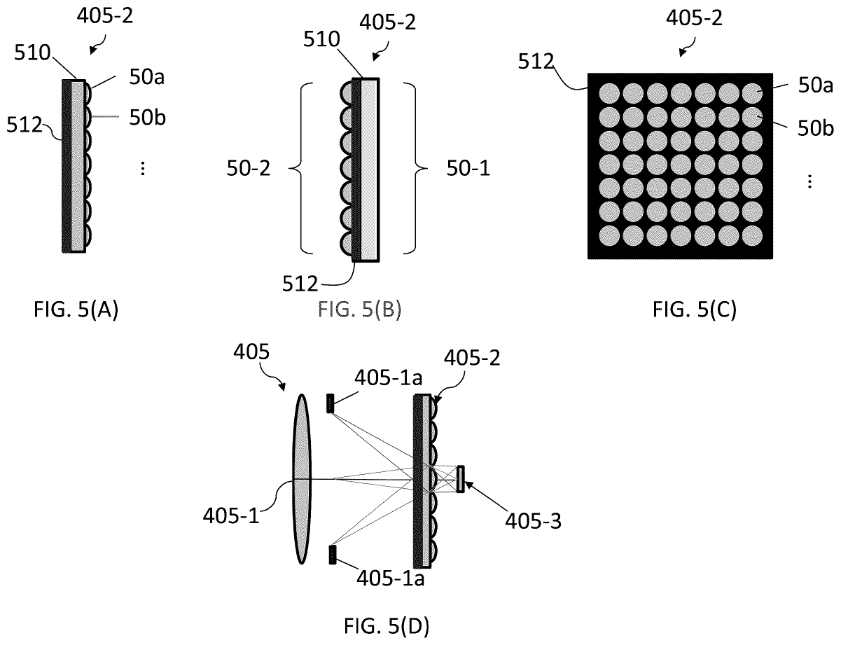

In the present invention, a new design of the light homogenizer is proposed to improve the uniformity of irradiance within individual zone of the multichannel array sensor. According to one embodiment of the present invention, FIG. 5(A) illustrates a cross-sectional view of a light homogenizer 405-2, which includes a substrate 510 with a plurality of microlenses (50a, 50b, . . . ) fabricated on one side of the substrate 510 to form a microlens array and an aperture stop layer 512 formed on the other side of the substrate 510. In an alternative embodiment, as shown in FIG. 5(B), the light homogenizer 405-2 includes a substrate 510 with a first plurality of microlenses 50-1 fabricated on one side of the substrate 510, an aperture stop layer 512 formed on the same side of the substrate 510. The aperture stop layer 512 includes a plurality of transparent zones to form an aperture array. FIG. 5(C) illustrates front view of the light homogenizer 405-2, where the aperture stop layer 512 is formed by black photoresist or other opaque materials on either side or both side of the substrate and is used to reduce the crosstalk and stray light between the plurality of transparent zones. FIG. 5(D) schematically illustrates light path of the imaging lens 405-1 being projected onto the multichannel array sensor 405-3, the image of the exit pupil 405-1a of the imaging lens 405-1 is formed at the sensor plane of the multichannel array sensor 405-3 by each microlens, where the exit pupil arranged between the imaging lens 405-1 and the microlens array of the light homogenizer 405-2. Uniformity and crosstalk between adjacent zones of the multichannel array sensor 405-3 are controlled by the size of aperture's opening of the aperture stop layer 512 and the distance between the imaging lens 405-1 and the microlens array.

According to embodiments of the present invention, the microlens array is formed by lenses with only one side has spherical shape.

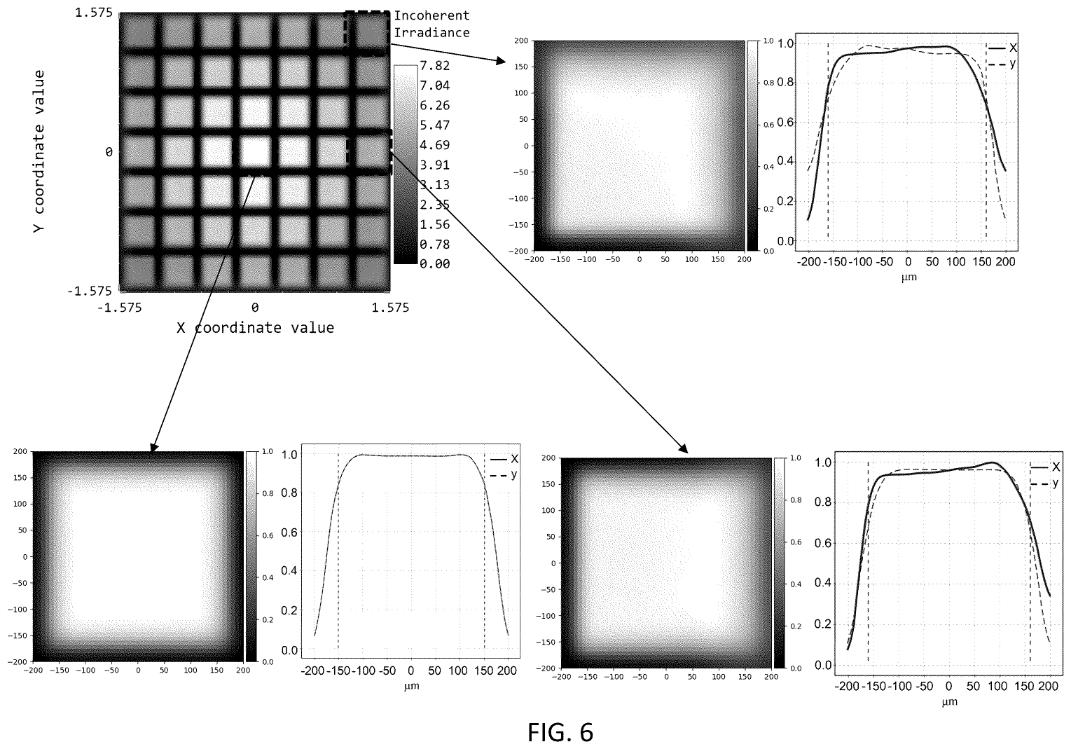

FIG. 6 illustrates irradiance on multichannel array sensor of the multichannel sensor module shown in FIG. 4(C) at various zone locations. Upper left of FIG. 6 shows the full irradiance map of the multichannel array sensor 405-3, while lower left, upper right and lower right of FIG. 6 are respectively showing irradiance maps of center zone, corner zone and edge zone. At the center zone, x-curve and y-curve are overlapping. Based on the results of FIG. 6, the irradiance transmittance and uniformity at various zone locations are greatly improved by utilizing the microlens array on the light homogenizer.

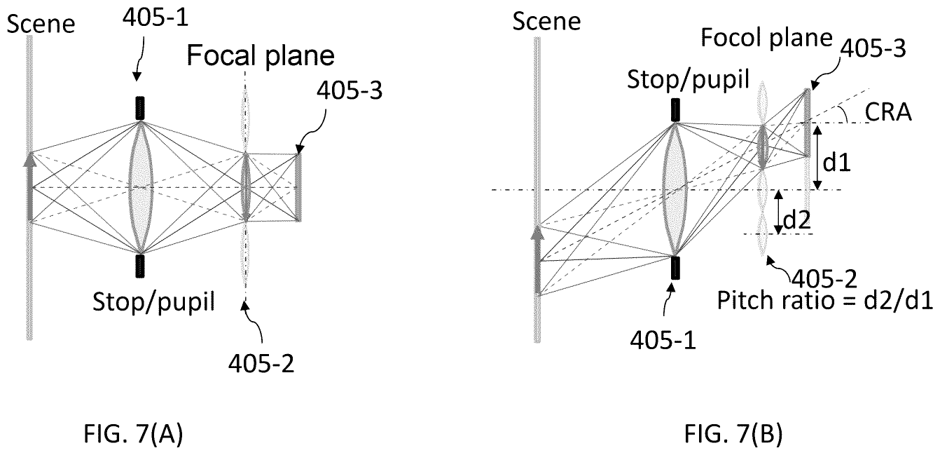

FIG. 7(A) and FIG. 7(B) are respectively illustrating axial light path and off-axial light path of microlens array of the light homogenizer 405-2 proposed in the present invention. In both cases, the microlens array of the light homogenizer 405-2 projects the images of the imaging lens 405-1 from different sub-scenes onto corresponding zones of the multichannel array sensor 405-3 directly without imaging processing. The spectral information of a sub-scene would be uniformly distributed within the corresponding zone of the multichannel array sensor 405-3 for generating irradiance with good uniformity.

According to embodiments of the present invention, the microlens array of the light homogenizer 405-2 is placed at focal plane of the imaging lens 405-1.

According to embodiments of the present invention, a ratio of a microlens pitch d2 to a zone pitch d1, i.e. d2/d1, is between 0.45 and 1.

According to embodiments of the present invention, the maximum chief ray angle (CRA) of the imaging lens is equal or less than 15 degrees.

According to embodiments of the present invention, a ratio of a microlens focal length to a zone pitch is between 2 and 8.

In summary, an integrated multichannel endoscope system proposed in the present invention, which utilizes the microlens array to replace diffuser, together with the use of imaging lens can directly mapping the irradiance distribution of the scene to a multichannel array sensor of the endoscope system with high fidelity and uniformity.

While various embodiments of the present invention have been described above, it should be understood that they have been presented by a way of example and not limitation. Numerous modifications and variations within the scope of the invention are possible. The present invention should only be defined in accordance with the following claims and their equivalents.

1. A multichannel integrated endoscope system, comprising:
a lighting module configured to emit illumination lights on an object;
an image module configured to capture an image of said object;
a multichannel sensor module, configured to obtain a spectral information of said object, including an imaging lens, a light homogenizer and a multichannel array sensor, wherein said light homogenizer is formed between said multichannel array sensor and said imaging lens.

2. The multichannel integrated endoscope system of claim 1, wherein said multichannel array sensor includes a plurality of spectral sensing zones.

3. The multichannel integrated endoscope system of claim 2, wherein each of said plurality of spectral sensing zones divides sensing field of view of said subject into sub field of views.

4. The multichannel integrated endoscope system of claim 2, wherein each of said plurality of spectral sensing zones includes a plurality of spectral-filter channels, and each of said plurality of spectral sensing zones generate the spectral information of said sub field of views.

5. The multichannel integrated endoscope system of claim 1, wherein said light homogenizer is a microlens array.

6. The multichannel integrated endoscope system of claim 5, wherein said microlens array is formed by lenses which only one side has spherical shape.

7. The multichannel integrated endoscope system of claim 5, wherein a ratio of a microlens pitch to a zone pitch is between 0.45 and 1.

8. The multichannel integrated endoscope system of claim 5, wherein a ratio of a microlens focal length to a zone pitch is between 2 and 8.

9. The multichannel integrated endoscope system of claim 5, wherein said microlens array includes:
a substrate;
a first plurality of microlenses formed on one side of said substrate; and
an aperture stop layer formed on same side or on other side of said substrate, wherein said aperture stop layer includes a plurality of transparent zones.

10. The multichannel integrated endoscope system of claim 9, wherein said aperture stop layer is form by black photoresist and used to reduce the crosstalk and stray light between said plurality of transparent zones.

11. (canceled)

12. A multichannel sensor module, comprising:
an imaging lens configured to receive light reflected from an object;
a light homogenizer configured to homogenize said light reflected from said object; and
a multichannel array sensor configured to receive light reflected from said object for obtaining spectral information of said object, wherein said light homogenizer is formed between said multichannel array sensor and said imaging lens.

13. The multichannel sensor module of claim 12, wherein said multichannel array sensor includes a plurality of spectral sensing zones.

14. The multichannel sensor module of claim 13, wherein each of said plurality of spectral sensing zones divides sensing field of view of said object into sub field of views.

15. The multichannel sensor module of claim 14, wherein each of said plurality of spectral sensing zones includes a plurality of multichannel filter array, and each of said plurality of spectral sensing zones generate the spectral information of said sub field of views.

16. The multichannel sensor module of claim 15, wherein each of said plurality of multichannel filter array includes a plurality of spectra of channels.

17. The multichannel sensor module of claim 12, wherein said light homogenizer is a microlens array.

18. The multichannel sensor module of claim 12, wherein said microlens array is form by lenses which only one side has spherical shape.

19. The multichannel sensor module of claim 17, wherein a ratio of a microlens pitch to a zone pitch is between 0.45 and 1.

20. The multichannel sensor module of claim 17, wherein a ratio of a microlens focal length to a zone pitch is between 2 and 8.

21. The multichannel sensor module of claim 17, wherein said microlens array includes:
a substrate;
a first plurality of microlenses formed on one side of said substrate; and
an aperture stop layer formed on other side of said substrate, wherein said aperture stop layer includes a plurality of transparent zones.

22. The multichannel sensor module of claim 17, wherein said aperture stop layer is formed by black photoresist and used to reduce the crosstalk and stray light between said plurality of transparent zones.

23. The multichannel sensor module of claim 21, wherein a second plurality of microlenses are formed on said aperture stop layer.
